(19) United States
(12) Patent Application Publication
(10) Pub. No.: US 2016/0338616 A1
(43) Pub. Date: Nov. 24, 2016

(54) MEDICAL DEVICE FOR DETERMINING COMPONENTS OF THE EXPIRATION VOLUME

(71) Applicant: FILT Lungen- und Thoraxdiagnostik GmbH, Berlin (DE)

(72) Inventor: Ruediger Eichler, Zellingen (DE)

(21) Appl. No.: 15/153,849

(22) Filed: May 13, 2016

(30) Foreign Application Priority Data
May 20, 2015 (DE) 20 2015 003 822.0

(51) Int. Cl. A61B 5/08; A61B 5/087; A61M 11/00; A61B 5/097

(52) U.S. Cl. CPC A61B 5/082; A61B 5/097; A61B 5/087; A61M 11/00

(57) ABSTRACT

A medical apparatus for determining constituents of expiration volume, using a through-flow sensor for determining constituents of expiration volume, designed as an interchangeable component in order to permit a simple and rapid exchange of the through-flow sensor with its sensor holder without having to dismantle the medical apparatus. The apparatus can thus be used for determining a very wide range of analytes (constituents of the exhalation). Preferred analytes are biomarkers in the breath condensate such as H₂O₂, leukotrienes, prostaglandins or interferons as indicators for diseased changes in the lung.

The invention relates to a medical apparatus for determining constituents of expiration volume using a through-flow sensor and a through-flow measuring unit.

Medical apparatuses of this type are known.

In WO 2008/106 961 A2, a transportable pneumotachograph is described to determine constituents of the expiration volume, which comprises a processor, a PEEP valve attached on the exhalation side on the pneumotachograph, a filter attached on the inhalation side on the pneumotachograph for removing the portion of the constituent to be determined in the inspiration air, at least one sensor attached in or on the pneumotachograph pipe, wherein with a sensor arranged on the pneumotachograph pipe, the pneumotachograph pipe comprises an opening on the sensor side and/or at least one means installed in the pneumotachograph pipe for removing samples, and further an optical and/or acoustic control of the expiration volume.

A correlation is enabled between recorded measured data from one or more constituents of the exhalation flow and a lung function test, wherein the analysis can be conducted taking into account standardised guidelines and enables a localisation of the seat of the disease.

A pneumotachograph is known from DE 10 2010 014 008 A1 with extended functionality, whereby in order to isolate analytes contained at least in the breath condensate of an exhalation in at least one flow channel, which has an inlet and an outlet for the exhalation, at least one carrier structure is arranged for capturing and collecting the breath condensate and/or at least one sensor is arranged for the quantitative and/or qualitative determination of at least one analyte contained in the breath condensate with electrodes, wherein the at least one carrier structure for capturing and collecting the breath condensate is exchangeable in the flow channel, the carrier structure and/or the sensor with its at least one carrier structure are permeable to fluid at least at times, so that they can be flowed through by the exhalation containing the breath condensate, and the carrier structure and/or the sensor with its at least one carrier structure with their respective enclosures on the circumference side fill out the profile of the flow channel over its entire surface.

The object of the invention is to optimise the method of operation of a medical apparatus of this type with a through-flow sensor for determining constituents of expiration volume through measures on the apparatus side and control technology measures, and to automate the method of functioning to a high degree.

This object is attained by means of the features described in apparatus claim 1. Advantageous designs are the subject of subclaims.

The medical apparatus according to the invention for determining constituents of expiration volume comprises at least the following apparatus components that are in fluid communication with each other:
on the patient side an air distributor comprising at least one air inlet for filtered ambient air which is closable via a valve, a connection to a breathing tube with a mouthpiece, wherein in the breathing tube or in the connection from the air distributor to the breathing tube, a closure means is arranged which can at least be moved into the “closed breathing tube” and “opened breathing tube” positions, and an outlet for the entire expiration volume,
a through-flow sensor installed on the outlet of the air distributor or in a flow guide tube connected to the outlet of the air distributor, and arranged in a sensor holder for determining at least one constituent of the expiration volume,
a flow generator arranged in the direction of flow behind the through-flow sensor at least for the controlled suctioning of the entire expiration volume through the through-flow sensor,
a through-flow measuring unit for determining the fluid flow flowing through the through-flow sensor,
a pressure measuring unit in front of or behind the through-flow sensor for determining an activation pressure for the flow generator,
a liquid dispensing apparatus for emitting or generating aerosol mist in the direction of flow in front of the through-flow sensor, and
a control unit at least for controlling the flow generator and the liquid dispensing apparatus. The interior of the air distributor preferably comprises a low volume in order to keep the dead volume which is not included in the measured value recording as low as possible.

The sensor holder with the through-flow sensor as a structural unit is preferably designed as an interchangeable component in order to permit a simple and rapid exchange of the through-flow sensor with its sensor holder without having to dismantle the medical apparatus.

In this manner, the apparatus can be used for determining a very wide range of analytes (constituents of the exhalation). Preferred analytes are biomarkers in the breath condensate such as H₂O₂, leukotrienes, Prostaglandins or Interferons as indicators for diseased changes in the lung.

The through-flow sensor is preferably a sensor selected from the group “electrochemical sensors, biochemical sensors, enzymatic sensors, chemiluminescence sensors, NO sensors, O₂ sensors, H₂O₂ sensors, CO₂ sensors, CO sensors” and/or a combination sensor of the known sensor types.

The through-flow sensor can additionally or as an alternative have ab- or adsorptive properties in order to filter out from the exhalation or to bind a constituent (biomarker) to be determined for subsequent external analyses.

The through-flow measuring unit can be a through-flow sensor of the known type or also a pneumotachograph.

In a further embodiment, in addition to the control unit, a display and computing unit can be installed. In the simplest embodiment, this is a PC or a tablet PC with a user interface with known operations such as programming and operating the apparatus, as well as recording measurements, calculating measured values and issuing measured values.

In the control unit and/or in the display and computing unit, programmes or programme modules can additionally be deposited for the process of individual or several method steps of the operating method, and for the operation and recording of measured values of different through-flow sensors, and stored there in such a manner that they can be retrieved.

In a preferred embodiment, the medical apparatus according to the invention is designed as a manual apparatus. In this embodiment, the apparatus is essentially operated in a fully automated manner, whereby the operation of the apparatus does not have to be conducted via a PC or a tablet PC. The programmes and control elements needed to record the measured values are here integrated into the control unit, so that the patient can record the measured values independently.

The flow generator, here an active one, which generates a fluid flow via a pump effect, can be housed in a separate enclosure. For a connection to the flow lines of the medical apparatus, the flow generator or its enclosure can be equipped with an adaptor with a preferably hose-shaped feed line, which is attached on the end side to the flow line pipe or to the outlet opening of the sensor holder. The adaptor closes the above-named connections in an airtight manner on the surrounding side.

The flow generator fulfils several functions. Its main function is to generate in the exhalation phase a defined, essentially constant fluid flow of the full exhalation through the through-flow sensor, which is preferably maintained beyond the exhalation phase in order to rinse the through-flow sensor with filtered ambient air on completion of an exhalation in order to remove the exhalation.

Additionally, the flow generator is used during the temperature adjustment of the through-flow sensor whereby the aerosol mist generated by the liquid dispensing apparatus in front of the through-flow sensor is suctioned out through the through-flow sensor and tempers said through-flow sensor through humidification.

This can occur simultaneously with the rinsing process between consecutive expiration phases, or as a separate measure.

Further, the flow generator is operated with filtered ambient air during calibration and to specify the zero point of the measuring system.

The function of the flow generator is to pull the exhalation out of the air distributor through the through-flow sensor, in which a detectable reaction of the constituent to be analysed takes place. Alternatively, the constituent to be analysed can be bound on the surface of the through-flow sensor and then be quantitatively and/or qualitatively analysed in a subsequent verification procedure.

The flow generator is in the simplest embodiment a pump, the pump capacity of which can be controlled and regulated.

The flow through the through-flow sensor is 20 ml/s to 100 ml/s, preferably 30 ml/s to 70 ml/s, in particular 50 ml/s.

The liquid dispensing apparatus, by means of which the through-flow sensor temperature can be maintained at an almost constant value, is a nebulizer, sprayer or atomizer.

In particular when the liquid dispensing apparatus is a nebulizer, said apparatus has a separate air inlet for ambient air which is filtered and suctioned in via a pump, and the air enriched with liquid, controlled by the control unit, is blown in through a further air inlet into the air distributor and suctioned out by the flow generator through the through-flow sensor. Said sensor is hereby tempered.

Depending on the pump capacity of the pump, filtered ambient air is not necessarily required through the air inlet of the air distributor.

Preferably, with this embodiment, the valve of the air inlet into the air distributor controlled by the control unit is also motorically adjustable, alongside the closure means in the breathing tube or in the connection from the air distributor to the breathing tube.

If the liquid dispensing apparatus is a sprayer or an atomizer, it is provided in a preferred embodiment that these can be directly triggered via the control unit to release liquid into the air distributor, wherein the air can be fed through the air inlet of the air distributor.

In the simplest case, distilled water is used as a liquid.

In a further advantageous design, a calibration unit is arranged on the air distributor, which has a separate air inlet for filtered ambient air which is suctioned in via a pump under the control of the control unit, and which comprises a separate inlet into the air distributor for emitting a calibration gas or a calibration fluid in front of the through-flow sensor.

The liquid dispensing apparatus and the calibration unit are preferably connected to a shared pump for filtered ambient air via a controllable switching valve.

The pressure measurement with a pressure measuring unit is used to detect a pressure increase the flow lines, preferably in the air distributor and/or in the flow guide tube, which is caused by the expiration pressure generated when the patient exhales and/or by the flow resistance of the through-flow sensor.

The flow resistance of the medical apparatus according to the invention, including of the electrochemical through-flow sensor, preferably generates a pressure level of 1 to 20 mbar, preferably 3 to 4 mbar.

Since preferably, the medical apparatus is closed to the surrounding environment on the side of the through-flow sensor located opposite the air distributor, when the valve in the air inlet of the air distributor is closed, no pressure compensation with the surrounding environment takes place, so that with the onset of exhalation, a continuous pressure increase can be registered.

Only when a defined inner pressure (activation pressure) in the air distributor or in the flow guide tube (pressure trigger threshold) is achieved or exceeded is the flow generator switched on. The control and regulation of the flow generator is conducted by means of the control unit, which processes the identified pressure values of the pressure measuring unit and converts them into corresponding control and regulation signals in order to adjust the flow rate (pump capacity) on the flow generator, which it forwards to said flow generator.

Due to the pressure increase in the air distributor or in the flow guide tube initiated by the patient during the initial phase of exhalation, it is additionally achieved that the lung of the patient is sufficiently ventilated and opened, and the velum is closed as far as possible.

The differential pressure resulting from the pressure increase in the medical apparatus according to the invention, whereby the flow generator is switched on or activated, lies between 1 mbar and 7 mbar, preferably between 2 mbar and 5 mbar, in particular at 3 mbar. The differential pressure is described in the context below as activation pressure for the flow generator.

A pressure measuring unit can be a pressure sensor or a differential pressure measuring unit. The valve that closes the air inlet into the air distributor is in a preferred embodiment a one-way valve for letting in filtered ambient air, which closes independently as soon as inhalation has been completed, although at the latest with the onset of the exhalation air flow. With another embodiment, the valve that closes the air inlet can be motorically adjusted and controlled via the control unit.

In front of the air inlet of the air distributor, preferably an air filter, in particular a scrubber filter is installed, with which the constituents to be determined that are contained in the ambient air or other compounds which interfere with or can falsify the analysis of the exhalation can be filtered out.

The closure means in the breathing tube or in the connection from the air distributor to the breathing tube can be an adjustable flap, a valve, a closable diaphragm, preferably a ring diaphragm or another structural closure means for closing the flow tubes. In order to integrate the process-related control and regulation unit of the medical apparatus according to the invention, the closure means is preferably equipped with a servomotor which is connected to the control and regulation unit. Alternatively, a mechanical locking mechanism can be installed which is manually operable.

Through the closure of the breathing tube or air distributor connection to which the breathing tube is attached, it is prevented that while suctioning in filtered ambient air by means of the flow generator for rinsing purposes in the initialisation phase, or for regeneration of the through-flow sensor for subsequent measurements, unfiltered ambient air or breathing gases from the patient are also suctioned in through the breathing tube.

In order to increase the aeration of the lung (ventilation) and/or to increase the release of the aerosols contained in the lung which contain constituents, the medical apparatus according to the invention can be quipped with a vibration exciter and/or with a pressure modulator, with which the inspiration air flow is turned into vibrations and/or pressure waves during the inspiration phase.

Preferably, for the purpose of generating an inspiration air flow which is superimposed with vibrations and/or pressure waves, the vibration exciter and/or the pressure modulator are installed on or in the flow channels of the air distributor or of the breathing tube that lead to the respiratory paths.

In a preferred embodiment, the flow channels of the air distributor are arranged in a Y-shape, i.e. the outlet and the breathing tube and/or the connection for the liquid dispensing apparatus and the breathing tube each include an obtuse angle and the air inlet is set down at the side into the flow channel outlet breathing tube. This is one possible optimised flow channel arrangement in the air distributor in teams of flow technology.

In a particular embodiment, the medical apparatus according to the invention consists of individual apparatus component modules, which comprise connection means which correspond to each other, so that the medical apparatus can be modularly compiled from the necessary apparatus components depending on the type of application. Through this embodiment form, not only an exchange but also a modification to the sequence of the apparatus components or the removal of individual apparatus components can be conducted.

In particular, at least the sensor holder, the adaptor of the flow generator, the air distributor, the liquid dispensing apparatus, the calibration unit and the breathing tube as well as the vibration exciter are designed as solitary apparatus components which can be interconnected via detachable connection means. Detachable connections of this type can be screw, plug-in, latching or bayonet connection which are designed as sealed connections. The liquid dispensing apparatus and the calibration unit can also be designed as a single component together with the pump which supplies them.

The medical apparatus according to the invention is preferably operated in such a manner that it is controlled by a computer. The individual operating phases are preferably stored as separate programme modules in the control unit or in the computer (computer and display unit), which can be retrieved and started up as a whole or if required also separately. The individual operating phases will be explained below.

Before initialising the medical apparatus, first the sensor with sensor holder required for recording the constituent to be analysed is first connected to the medical apparatus.

In order to initialise the apparatus according to the invention, the closure means in the breathing tube or air distributor outlet is closed by bringing it into the “closed breathing tube” position. After closure, the measuring programme is started and the flow generator is switched on in order to rinse the at least one through-flow sensor, as a result of which filtered ambient air is suctioned through the through-flow sensor through the opened air inlet of the air distributor, preferably for a specified duration. The so-called rinse flow can be between 10 ml/s and 300 ml/s, depending on the design of the through-flow sensor. Preferably, the rinse flow is between 50 ml/s and 100 ml/s.

The zero point of the measuring system is then set with the flow generator switched off and the closure means closed.

The term “zero point of the measuring system” designates the adjustment and tuning in procedures of the measurement configuration through to the achievement of a constant state.

Then, preferably a check-up phase of the through-flow sensor begins, insofar as it is an electrochemical sensor. Here, the through-flow sensor is electronically activated and a tune-in procedure of the standby current is conducted. After a defined threshold value is reached, the programme switches to “measured value recording” mode.

At the start of the inspiration phase which follows initialisation, in which the patient breathes in through the medical apparatus, the closure means is brought into the “opened breathing tube” position and the valve in the air inlet, whether motorically controlled or in manual operation mode, is opened, so that the patient in the inspiration phase can breathe in filtered ambient air via the also opened air inlet of the air distributor.

During the inspiration phase of filtered ambient air, the flow generator remains switched off. At the start of the expiration phase that follows the inspiration phase, in which the patient breathes out into the medical apparatus, the valve of the air inlet is mechanically or motorically closed and the expiration airflow is fully guided through the outlet of the air distributor towards the at least one through-flow sensor.

After the start of the expiration phase, the flow generator is switched on when a defined activation pressure level is reached or exceeded. The activation pressure is determined by a pressure measuring apparatus, which can be installed in a flow channel on the downstream side in relation to the through-flow sensor, or in the air distributor or in the breathing tube on the upstream side in relation to the through-flow sensor, and the recorded values are forwarded to the control unit which triggers and activates the flow generator. The flow generator then generates the necessary fluid flow required for recording the measured values on and in the through-flow sensor.

The duration of the expiration phase is between 5 and 30 seconds, preferably between 10 and 20 seconds.

In addition, at least for the duration of the expiration phase, the through-flow measuring unit is used to determine the fluid flow (volume flow) flowing through the through-flow sensor, on the basis of which the calculation of the biomarker concentration per volume unit can be conducted.

At least the measured values of the through-flow sensors, the pressure measuring unit and the through-flow measuring unit are recorded by the control unit and processed by the control unit into control signals and/or regulation signals and/or display signals for the apparatus components.

In a further embodiment, some of the above-named operations can be conducted from a PC or tablet PC and results and procedures can be displayed by means of a display unit.

Preferably, following completion of the expiration phase, the closure means in the breathing tube or the air distributor outlet is brought into the “closed breathing tube” position, in order to then rinse the through-flow sensor with filtered ambient air, wherein the filtered ambient air is suctioned through the through-flow sensor by means of the flow generator.

If prior to or during the rinsing process by the liquid dispensing device in front of the through-flow sensor an aerosol mist is generated, the through-flow sensor is at the same time tempered through humidification, so that the through-flow sensor temperature has almost the same value as before the first expiration phase.

If a nebulizer is used, it preferably suctions in filtered ambient air through a separate air inlet by means of a pump and adds this as liquid. The activation of the pump is controlled by the control unit.

If the liquid dispensing device is a sprayer or an atomizer, the release of liquid into the air distributor into the rinse flow is directly triggered by the control unit.

Here, the sensor is tempered, freed from exhalation, the zero value is determined and the sensor is prepared for the next measured value recording procedure.

For the purpose of calibration, when the valve into the air distributor is opened, the closure means is closed and the liquid dispensing device is switched off, a calibration fluid or a calibration gas is released from the calibration unit in front of the through-flow sensor and is suctioned by the flow generator through the through-flow sensor together with filtered ambient air.

In order to increase lung ventilation and release lung aerosols, at the start of the inspiration phase or at a later time during the further progress of the inspiration phase, the inspiration airflow can be made to vibrate with a vibration exciter or a pressure modulator in order to generate a vibration of the lung, through which the aerosols in the lung which contain the constituents are released into the exhalation.

In order to avoid falsely positive measurement results, the filtered ambient air can be analysed for the content of one or more of the constituents to be determined and/or the content of interfering compounds which can interfere with or falsify the measurement. The analysis of the filtered ambient air is preferably conducted prior to the inspiration phase. In the analysis phase of the ambient air, the closure means in the breathing tube or air distributor outlet is closed by being brought into the “closed breathing tube” position.

When determining at least one concentration of a constituent to be determined which is not tolerable for the measuring system and/or a compound in the filtered ambient air which interferes with the recording of the measured values, a warning signal can be issued and/or the continuation of the procedure can be interrupted through process control.

Naturally, it is also possible to analyse unfiltered ambient air, whereby the filter arranged in the air distributor on the entry side is removed.

The invention will be explained below with reference to the figures, in which:

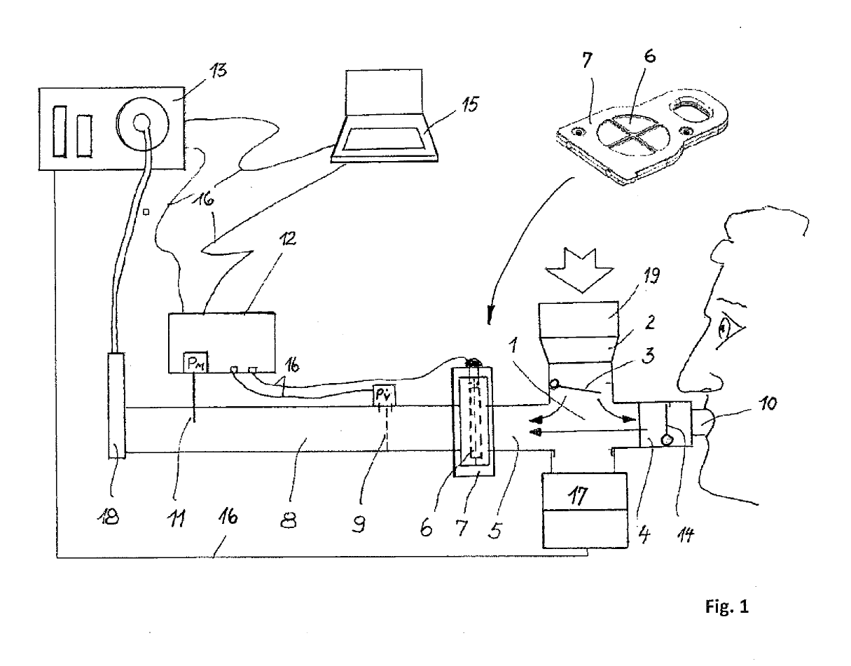

FIG. 1: shows a first embodiment of the medical apparatus,

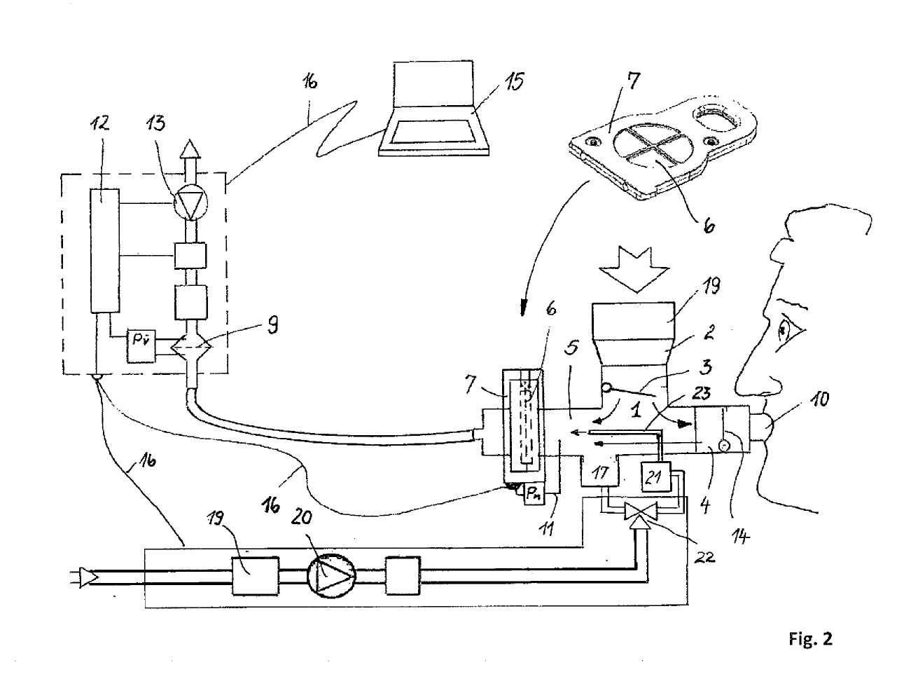

FIG. 2: shows a second embodiment of the medical apparatus, and

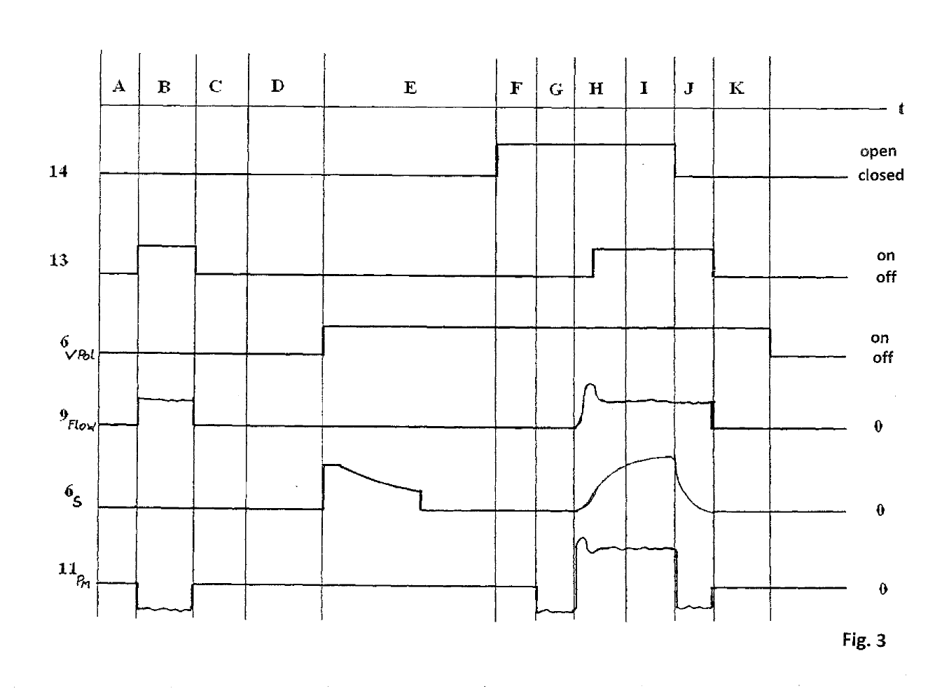

FIG. 3: shows the operating phases in chronological order with tempering of the through-flow sensor.

FIG. 1 shows a first embodiment of the medical apparatus for determining constituents (biomarkers) of expiration volume, whereby the apparatus components are in fluid communication with each other and a flow generator 13 is used. The medical apparatus according to the invention comprises an air distributor 1 which comprises an air inlet 2 for filtered ambient air which is closable via a valve 3, a connection to a breathing tube 4 with a mouthpiece 10 and an outlet 5.

In front of the air inlet 2 of the air distributor 1, a scrubber filter 19 is installed with which the constituents or other compounds contained in the ambient air which are to be determined, which can interfere with or falsify the analysis of the exhalation, are filtered out.

The through-flow sensor 6 for determining at least one constituent of the expiration volume forms a structural unit together with the sensor holder 7, which is installed in a flow guide tube 8 connected on the outlet 5 of the air distributor 1. The through-flow measuring sensor 6 extends over the entire profile surface of the flow guide tube 8.

In order to record the measured values and to control and regulate the through-flow sensor 6, said sensor has an apparatus interface 16 with a corresponding signal line to the control unit 12, in which the sensor-specific electronic components are integrated.

In the section of the flow guide tube 8 located in the downstream side in relation to the through-flow sensor 6, a pressure measuring unit 11 and a through-flow measuring unit 9 are installed for determining the volume flow flowing through the through-flow sensor, respectively comprising apparatus interfaces 16 with a corresponding signal line to the control unit 12.

The flow generator 13 is housed in a separate enclosure. For the connection to the flow guide tube 8 of the medical apparatus, the entrance to the flow generator enclosure is equipped with a hose-shaped feed line, on the end of which an adaptor 18 is installed, via which the flow generator 13 is connected to the end of the flow guide tube 8. The adaptor 18 closes the flow guide tube 8 on the surrounding side in an airtight manner.

The flow generator 13 can be connected to the control unit 12 via a further apparatus interface 16 with a corresponding signal line.

When a defined activation pressure (pressure trigger threshold) is reached or exceeded in the flow guide tube 8, the flow generator 13 is switched off. The control and regulation of the flow generator 13 is conducted via the control unit 12, which records the determined pressure values of the through-flow measuring unit 9, processes them and converts them into corresponding control and regulation signals for adjusting the flow rate (pump capacity) on the flow generator, which it forwards to said generator.

In the breathing tube 4, which is connected to the air distributor, a closure means 14 is arranged in the form of a flap, which is movable at least into the positions “closed breathing tube” and “opened breathing tube”.

Further, on the air distributor 1, a liquid dispensing device 17 is arranged. This is a sprayer or atomizer which controlled by the control unit 12 generates an aerosol mist in front of the through-flow sensor 6, which together with the ambient air from the air inlet 2 is suction out through the through-flow sensor 6 by the flow generator 13. The through-flow sensor 6 is thus tempered through humidification.

A vibration exciter, not shown, can be installed on the air distributor 1 for generating an inspiration flow over which vibration is superimposed.

For programming and operating the medical apparatus, and for displaying results and process sequences, in addition to the control unit 12, a PC 15 is installed with a keyboard and monitor.

FIG. 2 shows a second embodiment of the medical apparatus for determining constituents (biomarkers) of expiration volume with an active flow generator 13, in which the apparatus components are also in fluid communication with each other.

The medical apparatus according to the invention comprises an air distributor 1, which comprises an air inlet 2 for filtered ambient air which can be closed via a valve 3, a connection to a breathing tube 4 with a mouthpiece 10, and an outlet 5.

A scrubber filter 19 is installed in front of the air inlet 2 of the air distributor 1, with which the constituents or other compounds to be determined which are contained in the ambient air, and which interfere with or falsify the analysis of the exhalation, can be filtered out.

The through-flow sensor 6 for determining at least one constituent of expiration volume forms, together with the sensor holder 7, a structural unit, which is installed directly on the outlet 5 of the air distributor 1. The through-flow sensor 6 extends over the entire profile surface of the outlet 5.

In order to record the measured values and to control and regulate the through-flow sensor 6, said sensor has an apparatus interface 16 with a corresponding signal line to the control unit 12, in which the sensor-specific electronic components are integrated.

The flow generator 13 in the form of a pump is installed in a separate enclosure. This enclosure also contains the control unit 12 and the through-flow measuring unit 9.

For the connection to the sensor holder 7, the entrance to the flow generator enclosure is equipped with a hose-shaped feed line, via which the flow generator 13 is connected to the exit of the sensor holder 7.

The pressure sensor 11 is installed in the air distributor 1 in front of the through-flow sensor 6, and has an apparatus interface 16 with a corresponding signal line to the control unit 12 in the flow generator enclosure.

In the air distributor 1, a closure means 14 in the form of a flap is arranged in the connection area for the breathing tube 4, which is movable at least into the “closed breathing tube” and “opened breathing tube” positions. This is preferably achieved by means of a servomotor, which can be triggered by the control unit 12.

The liquid dispensing device 17 is here a nebulizer, which via a separate air inlet suctions in filtered ambient air by means of a pump 20 triggered by the control unit 12, and adds to this the liquid for generating an aerosol mist. This mist is then released into the air distributor 1 in the direction of flow in front of the through-flow sensor 6. With this embodiment, with corresponding pump volume of the pump 20, no additional filtered ambient air is required through the air inlet 2. Here, too the flow generator 13 suctions out through the through-flow sensor 6. The tempering of the through-flow sensor 6 through humidification can thus be conducted as a separate method step. Preferably, with this embodiment, the valve 3 of the air inlet 2 is also movable, via the servomotor which is triggerable via the control unit 12, into the position “closed air inlet 2” and “opened air inlet 2”.

Further, it is shown that a calibration unit 21 is arranged on the air distributor 1, which has a separate air inlet for filtered ambient air which is suctioned in via a pump 20, triggered by the control unit 12, and which comprises a separate inlet 23 into the air distributor 1 for releasing a calibration gas or calibration fluid in front of the through-flow sensor 6.

The liquid dispensing device 17 and the calibration unit 21 are connected to the shared pump 20 via a controllable switching valve 22 and can thus be mutually supplied with filtered ambient air.

A vibration exciter, not shown, is installed on the air distributor 1 for generating an inspiration flow over which vibration is superimposed.

For programming and operating the medical apparatus, and for displaying results and process sequences, in addition to the control unit 12, a PC 15 is installed with a keyboard and monitor. FIG. 3 shows examples of the operating phases and the function states set respectively and recorded measured value curves in the individual phases for the apparatus components for the closure means 14, active flow generator 13, pressure through-flow sensor 6, through-flow measuring unit 9 and pressure measuring unit 11.

The individual phases are:

### A Initialisation

### B Rinsing

C Zero point setting
D Check-up of the through-flow sensor 6, here an H₂O₂ sensor
E Tune-in procedure for the through-flow sensor 6 and correction
F Release of the medical apparatus for recording the measured values
G Inspiration phase
H Expiration phase
I End of the expiration phase at point in time Tₑₓ
J Cleaning and tempering of the through-flow sensor 6 by rinsing with filtered ambient air, adding a liquid in the form of an aerosol mist

### K Evaluation

The first three progression curves show the function states of the apparatus components for the closure means 14, active flow generator 13 and polarisation voltage of the through-flow sensor 6.

For the closure means 14, the positions “closed breathing tube”=“closed” and “opened breathing tube”=“opened” are shown. For the active flow generator 13, the states “switched off”=“off” and “switched on”=“on” are shown. The specified polarisation voltage of the through-flow sensor 6 is characterised by the abbreviations “switched off”=“off” and “switched on”=“on”.

The lower three curves show progression curves of recorded measured values for the fluid flow through the through-flow measuring unit 9, the through-flow sensor 6 and the pressure of the pressure measuring unit 11.

### LIST OF REFERENCE NUMERALS

1 Air distributor
2 Air inlet
3 Valve
4 Breathing tube
5 Outlet
6 Through-flow sensor
7 Sensor holder
8 Flow guide tube
9 Through-flow measuring unit
10 Mouthpiece
11 Pressure measuring unit
12 Control unit
13 Flow generator
14 Closure means
15 Computer and display unit
16 Interfaces for apparatus control and measured value recording
17 Liquid dispensing device
18 Adaptor of the flow generator
19 Air filter
20 Pump
21 Calibration unit
22 Switching valve
23 Inlet

1. A medical apparatus for determining constituents of expiration volume, comprising at least the following as apparatus components that are in fluid communication with each other
an air distributor (1) comprising at least one air inlet (2) for filtered ambient air which is closable via a valve (3), a connection to a breathing tube (4) with a mouthpiece (10), wherein in the breathing tube (4) or in the connection from the air distributor (1) to the breathing tube (4), a closure means (14) is arranged which can at least be moved into the “closed breathing tube” and “opened breathing tube” positions, and an outlet (5) for the entire expiration volume,
a through-flow sensor (6) installed on the outlet (5) of the air distributor (1) or in a flow guide tube (8) connected to the outlet (5) of the air distributor (1), and arranged in a sensor holder (7) for determining at least one constituent of the expiration volume,
a flow generator (13) arranged in the direction of flow behind the through-flow sensor (6) at least for the controlled suctioning of the entire expiration volume through the through-flow sensor (6),
a through-flow measuring unit (9) for determining the fluid flow flowing through the through-flow sensor (6),
a pressure measuring unit (11) in front of or behind the through-flow sensor (6) for determining an activation pressure for the flow generator (13),
a liquid dispensing device (17) for emitting or generating aerosol mist in the direction of flow in front of the through-flow sensor (6), and
a control unit (12) at least for controlling the flow generator (13) and the liquid dispensing device (17).

2. The medical apparatus according to claim 1, further comprising a display and computing unit (15).

3. The medical apparatus according to claim 1, wherein the sensor holder (7) with the through-flow sensor (6) is designed as an interchangeable component.

4. The medical apparatus according to claim 1, wherein the closure means (14) can be moved using a servomotor and under the control of the control unit (12).

5. The medical apparatus according to claim 1, wherein the liquid dispensing device (17) is a nebulizer, sprayer or atomizer.

6. The medical apparatus according to claim 1, wherein the liquid dispensing device (17) has a separate air inlet for filtered ambient air which is suctioned in via a pump (20) under the control of the control unit (12), and the air enriched with liquid can be blown into the air distributor (1) via a further air inlet, or the liquid dispensing device (17) can be directly triggered via the control unit (12) in order to emit liquid into the air distributor (1).

7. The medical apparatus according to claim 1, wherein a calibration unit (21) is arranged on the air distributor (1), which has a separate air inlet for filtered ambient air which is suctioned in via a pump (20) under the control of the control unit (12), and which comprises a separate inlet (23) into the air distributor (1) for emitting a calibration gas or a calibration fluid in front of the through-flow sensor (6).

8. The medical apparatus according to claim 7, wherein the liquid dispensing device (17) and the calibration unit (21) are connected to a shared pump (20) via a controllable switching valve (22).

9. The medical apparatus according to claim 8, wherein when the valve (3) is opened, the closure means (14) is closed and the liquid dispensing device (17) is switched off, the calibration liquid or calibration gas can be released from the calibration unit (21) in front of the through-flow sensor (6), and can be suctioned together with filtered ambient air by the flow generator (13) through the through-flow sensor (6) for calibration purposes.

10. The medical apparatus according to claim 1, wherein on or in the breathing tube (4) or on or in the air distributor (1), a vibration exciter and/or a pressure modulator is installed for generating an inspiration flow over which vibration and/or pressure is/are superimposed.

11. The medical apparatus according to claim 1, wherein the medical apparatus is comprised of apparatus component modules which comprise connection means that correspond to each other.
